(19) United States
(12) Patent Application Publication
(10) Pub. No.: US 2011/0245640 A1
(43) Pub. Date: Oct. 6, 2011

(54) Films For Touchscreen Displays Of Medical Device Monitors

(75) Inventor: Scott MacLaughlin, Windsor, CO (US)

(73) Assignee: Nellcor Puritan Bennett LLC, Boulder, CO (US)

(21) Appl. No.: 13/076,318

(22) Filed: Mar. 30, 2011

Related U.S. Application Data
(60) Provisional application No. 61/319,510, filed on Mar. 31, 2010.

(51) Int. Cl. A61B 5/1455; G06F 3/041; B05D 5/08

(52) U.S. Cl. 600/324; 345/173; 427/64

(57) ABSTRACT

According to various embodiments, a medical device monitor includes a touchscreen and a film disposed on an external surface of the touchscreen. The film resists formation of visible fingerprints and resists growth of microorganisms. In various embodiments, one layer or more than one layer of the film may be disposed on the touchscreen.

### BACKGROUND

The present disclosure relates generally to medical devices and, more particularly, to medical monitors.

This section is intended to introduce the reader to various aspects of art that may be related to various aspects of the present disclosure, which are described and/or claimed below. This discussion is believed to be helpful in providing the reader with background information to facilitate a better understanding of the various aspects of the present disclosure. Accordingly, it should be understood that these statements are to be read in this light, and not as admissions of prior art.

In the course of treating a patient, a medical monitor may be used to monitor one or more patient parameters of interest. The monitor may be connected to a sensor disposed on or in the patient and used to generate data processed by the monitor in the course of monitoring the parameters of interest. In addition, the monitor may have a touchscreen display, which may show readings generated by the monitor, and may provide an interface that enables the clinician to change or adjust measurement settings of the monitor. Such touchscreens allow the clinician to interact with the monitor by touching the screen.

Because of the nature of a clinical or hospital setting, a clinician may touch a variety of substances including blood, bodily fluids, or other materials. If not removed from the fingers or glove of the clinician, these substances may be transferred to the touchscreen and smear or smudge, interfering with readability. Even a clean finger may leave fingerprints that may accumulate with time to affect the ability of the clinician to read information from the touchscreen. Cleaning solutions and/or wipes to remove fingerprints and other substances may not be available or impractical to use on a frequent basis.

### BRIEF DESCRIPTION OF THE DRAWINGS

Advantages of the disclosed techniques may become apparent upon reading the following detailed description and upon reference to the drawings in which:

FIG. 1 illustrates a pulse oximetry system coupled to a multi-parameter patient monitor and a sensor according to various embodiments;

FIG. 2 illustrates a pulse oximeter, according to an embodiment;

FIG. 3 is a partial cross-sectional view of an exemplary touchscreen display with a single layer of fingerprint-resistant and antimicrobial film, according to an embodiment;

FIG. 4 is a partial cross-sectional view of an exemplary touchscreen display with multiple layers of fingerprint-resistant and antimicrobial film, according to an embodiment; and

FIG. 5 is a perspective view of an exemplary stack of multiple layers of fingerprint-resistant and antimicrobial film configured to facilitate individual removal of layers, according to an embodiment.

### DETAILED DESCRIPTION OF SPECIFIC EMBODIMENTS

One or more specific embodiments of the present techniques will be described below. In an effort to provide a concise description of these embodiments, not all features of an actual implementation are described in the specification. It should be appreciated that in the development of any such actual implementation, as in any engineering or design project, numerous implementation-specific decisions must be made to achieve the developers' specific goals, such as compliance with system-related and business-related constraints, which may vary from one implementation to another. Moreover, it should be appreciated that such a development effort might be complex and time consuming, but would nevertheless be a routine undertaking of design, fabrication, and manufacture for those of ordinary skill having the benefit of this disclosure.

Medical device monitors, such as pulse oximeters, are used in a variety of applications and settings. Increasingly, these monitors include touchscreen displays, which enable a clinician to interact with the monitor by touching the screen with a finger instead of moving physical switches and knobs. After repeated touching by gloved or ungloved fingers, such touchscreens can become difficult to read because of the accumulation of fingerprints, sebum (i.e. skin oils secreted by the sebaceous glands), other oils, dirt, dust, debris, blood, bodily fluids, or other materials that may be found in a clinical or hospital setting. Not only do such materials act directly as an optical barrier between the clinician and the touchscreen, but they may also smudge or smear creating an effect over a larger area. In addition, these materials may scratch the touchscreen, further inhibiting readability. Finally, some of these materials reflect light and may reflect more when smudged. Because of the high intensity light found in clinical and hospital settings, such added reflection can contribute to make touchscreens difficult to read. Furthermore, the blood and bodily fluids left on the screen may harbor microorganisms. In the particular embodiments described below, a fingerprint-resistant, antimicrobial film is applied to the surface of the touchscreen to reduce the effects described above.

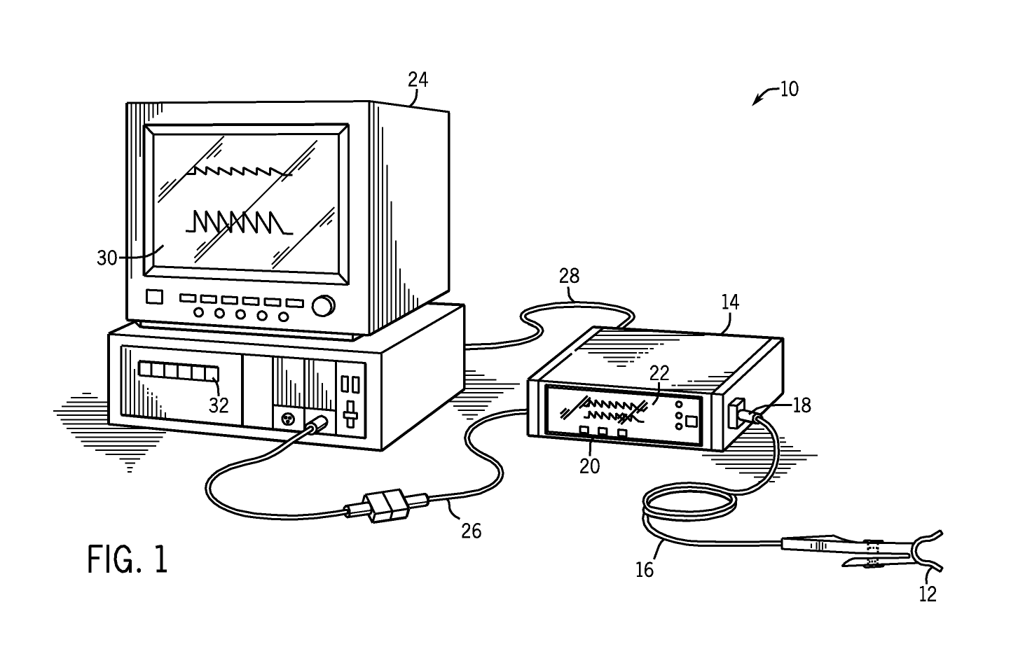

With the foregoing considerations in mind, FIG. 1 depicts a medical monitoring system 10 having a sensor 12 coupled to a monitor 14 in accordance with an embodiment of the present disclosure. The sensor 12 may be coupled to the monitor 14 via sensor cable 16 and sensor connector 18, or the sensor 12 may be coupled to a transmission device (not shown) to facilitate wireless transmission between the sensor 12 and the monitor 14. The monitor 14 may be any suitable monitor, such as those available from Nellcor Puritan Bennett LLC. The monitor 14 may be configured to calculate physiological parameters from signals received from the sensor 12 when the sensor 12 is placed on a patient. In some embodiments, the monitor 14 may be primarily configured to determine, for example blood and/or tissue oxygenation and perfusion, respiratory rate, respiratory effort, continuous non-invasive blood pressure, cardiovascular effort, glucose levels, level of consciousness, total hematocrit, hydration, electrocardiography, temperature, or any other suitable physiological parameter. Additionally, the monitor 14 may include a touchscreen display 20 configured to display information regarding the physiological parameters, information about the system, and/or alarm indications. The touchscreen 20 also enables a clinician to interact with the monitor 14. A fingerprint-resistant, antimicrobial film 22 covers the touchscreen 20.

To upgrade conventional operation provided by the monitor 14 to provide additional functions, the monitor 14 may be coupled to a multi-parameter patient monitor 24 via a cable 26 connected to a sensor input port or via a cable 28 connected to a digital communication port. In addition to the monitor 14, or alternatively, the multi-parameter patient monitor 24 may be configured to calculate physiological parameters and to provide a central display 30 for information from the monitor 14 and from other medical monitoring devices or systems. In some embodiments, the monitor 24 may be configured to display and/or determine some or all of the same physiological parameters as monitor 14. The monitor 24 may include various input components 32, such as knobs, switches, keys and keypads, buttons, etc., to provide for operation and configuration of the monitor 24. In addition, the monitor 14 and/or the multi-parameter patient monitor 24 may be connected to a network to enable the sharing of information with servers or other workstations.

The sensor 12 may be any sensor suitable for detection of any physiological parameter. The sensor 12 may include optical components (e.g., one or more emitters and detectors), acoustic transducers or microphones, electrodes for measuring electrical activity or potentials (such as for electrocardiography), pressure sensors, motion sensors, temperature sensors, etc. In one embodiment, the sensor 12 may be configured for photo-electric detection of blood and tissue constituents. For example, the sensor 12 may be a pulse oximetry sensor, such as those available from Nellcor-Puritan Bennett LLC. As shown in FIG. 1, the sensor 12 may be a clip-type sensor suitable for placement on an appendage of a patient, e.g., a digit, an ear, etc. In other embodiments, the sensor 12 may be a bandage-type sensor having a generally flexible sensor body to enable conformable application of the sensor to a sensor site on a patient. In yet other embodiments, the sensor 12 may be secured to a patient via adhesive (e.g., in an embodiment having an electrode sensor) on the underside of the sensor body or by an external device, such as headband or other elastic tension device. In yet other embodiments, the sensor 12 may be configurable sensors capable of being configured or modified for placement at different sites (e.g., multiple tissue sites, such as a digit, a forehead of a patient, etc.).

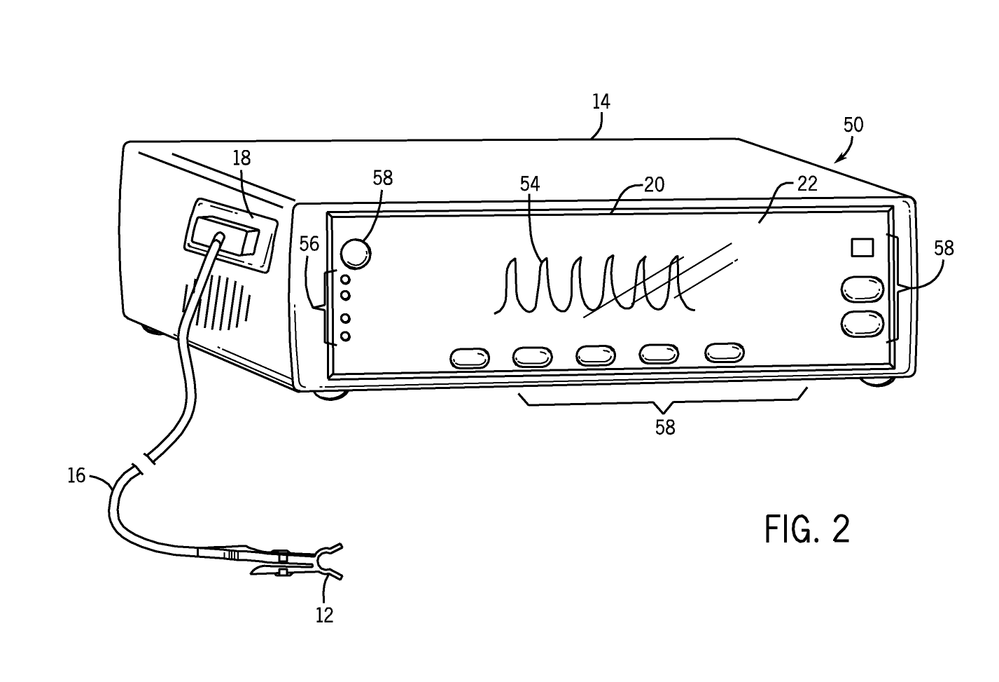

Turning to FIG. 2, a perspective view of a medical device monitor 14 is illustrated in accordance with an embodiment. In one embodiment, the medical device monitor 14 may be a pulse oximeter 50, such as those available from Nellcor Puritan Bennett LLC. The monitor 14 may be configured to display measured and/or calculated parameters on the touchscreen display 20. In certain embodiments, one area of the touchscreen 20 may be configured to display computed physiological data including, for example, an oxygen saturation percentage, a pulse rate, and/or a plethysmographic waveform 54. By touching the waveform 54 at a particular location, a user may be able to select a zoomed-in view at that time. In other embodiments, areas of the touchscreen 20 may display information related to alarms, monitor settings, and/or signal quality via indicator lights 56 built into the touchscreen. Touching near a light indicating an alarm may act to silence the alarm. In further embodiments, the touchscreen 20 may include multiple control areas 58 to facilitate user selections and input. The control areas 58 may be configured in different ways and may correspond to mechanical input devices, such as knobs, switches, or buttons. Unlike mechanical input devices, the control areas 58 may be responsive to the touch of a finger only. For example, to simulate a knob, the user may touch one part of the control area 58 with a finger and then slide the finger to represent turning the knob. Each of the control areas 58 may be labeled to indicate what function is performed when touched.

In one embodiment, the fingerprint-resistant, antimicrobial film 22 covers the entire area of the touchscreen 20, where a user is most likely to touch. In particular embodiments, the film 22 is transparent (i.e. optically clear) to reduce the effect of the film itself on readability of the touchscreen 20. Fingerprint-resistance may help maintain readability of the touchscreen 20 in spite of an accumulation of sebum and similar substances. A variety of technologies may be used to provide fingerprint-resistance. For example, in one embodiment, oleophobic and/or lipophobic materials added to the film 22 may provide fingerprint-resistance. Oleophobicity and lipophobicity are related concepts. Specifically, a material that is oleophobic repels oils and a lipophobic material is insoluble in lipids (i.e. fats) and thus does not adsorb lipids. Therefore, if the film 22 is oleophobic or lipophobic, it may repel sebum and similar oily substances, reducing the visibility of fingerprints. Examples of materials that may be incorporated into the film 22 to provide oleophobic and/or lipophobic properties include, but are not limited to, tetrafluoroethylene and perfluromethylvinylether. Using such materials, the film 22 may have an oil resistance of at least approximately 7 as determined by American Association of Textile Chemists and Colorists (AATCC) Test Method 118-2007 (revised). In another embodiment, a nanostructure on the external surface of the film 22 absorbs oils and spreads them into a thin layer. Such a thin layer of oil may be almost imperceptible to the clinician. An example of a material that may be fabricated as a nanostructure is fluorinated polyhedral oligomeric silsesquioxane. Adding up to approximately 0.1 mL of n-heptane to an approximately 3 cm square, 0.15 mm thick film 22 with such a nanostructure may enable 12 point text on a sheet of paper held behind and against the film to be clearly read from a distance of 30 cm.

Not only does the build-up of foreign substances on the touchscreen 20 pose a readability problem, it may also act as a route for microbe transmission or as a medium for microbe growth. A clinician working with a patient may be exposed to a fluid, such as blood, containing microorganisms that may be inadvertently transferred to the touchscreen 20. The microorganisms may remain viable on the surface of the touchscreen 20 for some time unless it is disinfected. The touchscreen 20 may thus harbor undesired microorganisms absent preventative action.

In light of this, in one embodiment, the film 22 also resists the growth of microorganisms or kills microorganisms. A variety of materials possesses antimicrobial properties and may be suitable for use in the film 22. For example, in one embodiment, silver-based additives in the film 22 may be able to kill bacteria. In other embodiments, polymers provided in the film 22 may be able to penetrate the cell wall of a microorganism and thereby interfere with the capability of the microorganism to function, grow, or reproduce. Examples of these polymers include, but are not limited to 3 to 8 unit PVP chains, such as hexyl-PVP. In one embodiment, these polymers work by leaching out of the film 22 and into the microorganisms. In other embodiments, the structure of polymers in the film 22 may be such that the cell walls of microorganisms are punctured and ruptured, thereby killing them. Examples of these polymers include, but are not limited to, cationic quaternary ammonium salt, chlorhexidine (biguanide), and fluoroquinoline. These polymers are not designed to migrate out of the film 22 or rub off the surface of the film. Using such polymers, the film 22 may have a zone of inhibition of at least approximately 4 mm for common bacteria such as Salmonella and Escherichia coli as determined by AATCC Test Method 147-2004.

Moreover, the film 22 may be configured with additional beneficial properties for clinical or hospital settings. For example, in one embodiment, the film 22 may resist scratching to reduce the loss of readability caused by dirt and other abrasive substances. In other embodiments, the film 22 may reduce glare to counteract the effect of substances that reflect light. In further embodiments, a first material in the film 22 may provide the fingerprint-resistant property and a second material may provide the antimicrobial property. Each material may be placed in a separate sublayer of the film 22 or mixed together in a single layer. Alternatively, in other embodiments, a single material may possess both properties.

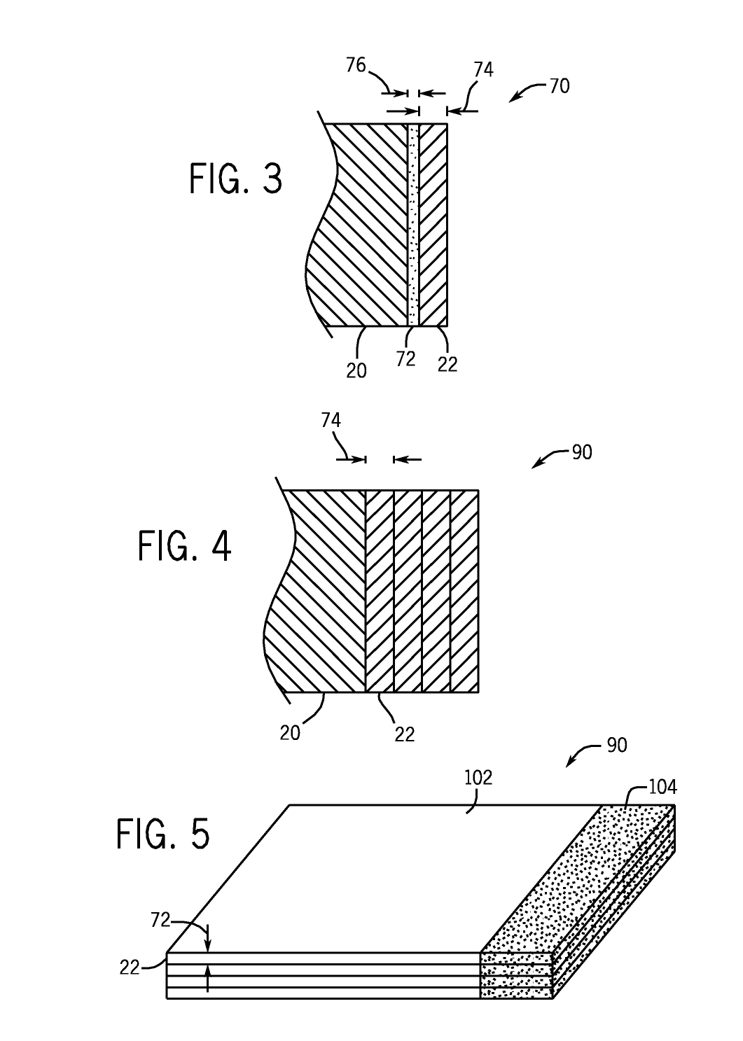

Different methods may be used to adhere the film 22 to the touchscreen 20. For example, looking more closely at the touchscreen 20 of the pulse oximeter 50, FIG. 3 shows a partial cross-sectional view of one embodiment of a single layer 70 of film 22 covering all or part of the external surface of the touchscreen 20. In one embodiment, an adhesive backing 72 may be used to adhere the film 22 to the touchscreen 20. The tackiness of the adhesive backing 72 may be configured such that it helps retain the film in place, but does not make removal difficult or leave behind an undesirable residue. Examples of such adhesive backings 72 include, but are not limited to, tackifier resins such as rosins, terpenes, aliphatic, cycloaliphatic, hydrogenated hydrocarbon, and terpene-phenol. Such adhesive backings 72 may have coefficient of friction values of at least approximately 1.5. In one embodiment, the film 22 has a thickness 74, which in certain embodiments may be between approximately 0.1 and 0.2 mm. The adhesive backing 72 has a thickness 76, which in certain embodiments may be between approximately 0.05 and 0.1 mm. In alternative embodiments, static electricity may be used to adhere the film 22 to the touchscreen 20. Such a method enables the film 22 to be removed and reapplied many times and leaves no appreciable residue. Other suitable methods of adhering films to surfaces may also be used. By covering all or part of the surface of the touchscreen 20, the film 22 helps to reduce the negative effects of fingerprints on the touchscreen and decreases the incidence and/or growth of microorganisms.

FIG. 4 is a partial cross-sectional view of a stack 90 of multiple layers of fingerprint-resistant, antimicrobial film 22 covering all or part of the external surface of the touchscreen 20. More than one layer of film 22 may be useful if the outermost layer of film is damaged from extended use or loses its effectiveness. The user may then remove the outermost layer, exposing an unused layer of film 22 underneath. When the last layer of film 22 is removed, a new stack 90 of layers of film may be adhered or attached to the surface of the touchscreen 20. As shown in FIG. 4, static electricity may be used to adhere adjacent layers of film 22 to each other. Alternatively, an adhesive backing similar to that shown in FIG. 3 may be used. Similarly, either static electricity or an adhesive backing may be used to adhere the stack 90 of layers of film 22 to the touchscreen 20.

In order to show how layers of film 22 may be removed, FIG. 5 is a perspective view of a stack 90 of multiple layers of fingerprint-resistant, antimicrobial film 22. In one embodiment, each layer of film 22 is composed of two regions. An adhesive region 102 is configured to be large enough to cover most or all of the area of the touchscreen 20. The adhesive region 102 may utilize an adhesive backing or static electricity as described above. A separation region 104 is configured to be non-adhesive and large enough for a person to able to separate one layer of film 22 from another by pulling or grabbing the region. The separation region 104 is configured not to use an adhesive backing or may be coated to prevent static cling. Because the separation regions 104 do not adhere to each other, the user may be able to remove one layer of film 22 at a time. In addition, the user may be able to remove the entire stack 90 of layers of film 22 by pulling or grabbing the separation region 104 of the bottommost layer of film. The adhesive regions 102 and separation regions 104 may have the same thickness 72 as shown or they may be different. Other configurations commonly used to enable individual separation of layers of film may also be used.

The specific embodiments described above have been shown by way of example, and it should be understood that these embodiments may be susceptible to various modifications and alternative forms. It should be further understood that the claims are not intended to be limited to the particular forms disclosed, but rather to cover all modifications, equivalents, and alternatives falling within the spirit and scope of this disclosure.

What is claimed is:

1. A medical device monitor comprising:
a touchscreen; and
a transparent film disposed on an external surface of the touchscreen, wherein the transparent film resists formation of visible fingerprints and resists growth of microorganisms.

2. The medical device monitor of claim 1, wherein the transparent film comprises a first material that resists formation of visible fingerprints and a second material that resists growth of microorganisms.

3. The medical device monitor of claim 1, wherein the transparent film adheres to the external surface of the touchscreen via static electricity or via an adhesive backing.

4. The medical device monitor of claim 1, wherein the transparent film is oleophobic, lipophobic, or a combination of both.

5. The medical device monitor of claim 1, wherein the transparent film comprises a nanostructure such that oils are absorbed and spread into a layer on an external surface of the transparent film.

6. The medical device monitor of claim 1, wherein the transparent film resists smudges from oil, blood, bodily fluids, dirt, or any combination thereof.

7. The medical device monitor of claim 1, wherein the transparent film comprises a composition that leaches into microorganisms.

8. The medical device monitor of claim 1, wherein the transparent film comprises a composition that ruptures cell walls of microorganisms.

9. The medical device monitor of claim 1, wherein the transparent film comprises a silver-based compound.

10. The medical device monitor of claim 1, wherein the transparent film resists scratching.

11. The medical device monitor of claim 1, wherein the transparent film reduces glare.

12. The medical device monitor of claim 1, wherein a thickness of the transparent film is between approximately 0.1 and 0.2 millimeters.

13. A pulse oximetry system comprising:
a pulse oximetry monitor capable of receiving signals generated by a pulse oximetry sensor, the pulse oximetry monitor comprising:
a touchscreen; and
one or more layers of film disposed on an external surface of the touchscreen, wherein each layer of the film comprises a first material that resists formation of visible fingerprints and a second material that resists growth of microorganisms.

14. The pulse oximetry system of claim 13, wherein each layer of film comprises a separation region to enable one layer of film to be removed from the touchscreen at a time.

15. The pulse oximetry system of claim 13, wherein the film adheres to the external surface of the touchscreen and to other layers of film via an adhesive backing or via static electricity.

16. The pulse oximetry system of claim 13, wherein the first material of each layer of the film is disposed in a first sublayer and the second material is disposed in a second sublayer.

17. The pulse oximetry system of claim 13, wherein the first material comprises a nanoscale material that absorbs and/or spreads oils to reduce the visible display of fingerprints on the touchscreen.

18. The pulse oximetry system of claim 13, wherein the second material comprises a polymeric composition capable of killing microorganisms.

19. The pulse oximetry system of claim 13, wherein each layer of the film is oleophobic and/or lipophobic.

20. A method of coating a touchscreen of a medical device monitor, the method comprising:
disposing a transparent film on an external surface of a touchscreen of a medical monitor, wherein the film resists formation of visible fingerprints and resists growth of microorganisms.
